(19) United States
(12) Patent Application Publication
(10) Pub. No.: US 2007/0092925 A1
(43) Pub. Date: Apr. 26, 2007

(54) Method of treating skin by delivering specific protein stabilizers/protectors to skin proteins and methods of selecting said stabilizers

(75) Inventor: Daniel Thorn Leeson, New York, NY (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/258,257

(22) Filed: Oct. 25, 2005

(51) Int. Cl. C12Q 1/37

(52) U.S. Cl. 435/23

(57) ABSTRACT

The invention provides methods of treating skin by delivering specific protein stabilizers/protectors to skin proteins, as well as to methods of selecting said stabilizer.

### FIELD OF THE INVENTION

The invention relates to skin care and cleansing formulations for treating skin and/or skin conditions (e.g., dry skin, psoriasis). More specifically, the invention relates to methods of treating skin by delivering protein stabilizing compounds to skin proteins which proteins may, for example, either be integral to skin structure (e.g., keratin) and/or regular skin function (e.g., enzymes regulating desquamation). The invention further relates to methods of selecting which of said protein stabilizers should be used.

### BACKGROUND OF THE INVENTION

Human skin is a very active and complex environment. The outermost layer of the skin, known as the stratum corneum, protects the body against penetration of exogenous compounds while also protecting against loss of moisture.

The stratum corneum renews itself roughly about every 30-40 days. The stratum corneum is made up of flattened cells called corneocytes. Corneocytes in turn are largely made up of the protein keratin which is a structural protein. Disruption of keratin structure would have a detrimental effect on skin. Identifying and providing compounds that protect/stabilize keratin (e.g., against protein denaturation) would thus be greatly beneficial.

As indicated, the stratum corneum, largely made up of corneocytes, is constantly renewing itself. New corneocytes are being generated at the base of the stratum corneum, while matured corneocytes are released at the skin surface. This process is regulated by enzymes (a type of protein) that digest the protein linkages between the corneocytes, said linkages generally being referred to as desmosomes. Therefore, to maintain a healthy outer layer of the skin requires optimal function of said enzymes. This stability of enzymes may in turn be compromised by environmental factors such as temperature, pressure and humidity. Examples of challenges to skin enzymes are (1) low water levels (caused, for example, by low ambient humidity or disruption of lipid bilayers); and (2) binding of exogenous compounds (e.g., surfactants found in cleansing products).

While not wishing to be bound by theory, it is believed that certain skin ailments such as xerosis (dry skin), and more serious conditions, such as psoriasis, are associated with reduced enzyme active. Again, while not wishing to be bound by theory, it is believed that reduction in enzyme activity (specifically for the enzymes involved in digesting the protein links between corneocytes) causes incomplete desmosome digesting leading to accumulation of “flakes” consisting of large clumps of corneocytes on the skin surface.

Thus, analogous to the case with the structural keratin proteins found in corneocytes (where protecting/stabilizing keratin helps skin), identifying and providing compounds that protect/stabilize enzymes (in this case the desmosome digesting enzyme) would be greatly beneficial.

Applicants have now made various important and unexpected discoveries. In one embodiment, applicants have discovered a method/test for selecting compounds which serve as protein stabilizers/protectors (e.g., protecting structural compounds such as keratin and/or desmosome digesting enzymes responsible for skin health. In a second embodiment, applicants have discovered a method for treating skin (e.g., treating ailments such as psoriasis associated with denaturing of desmosome digesting enzymes; or treating to prevent denaturing of structural proteins found in corneocytes) delivering specific protein stabilizers/protectors to the skin. In a third embodiment, applicants' invention is directed to compositions comprising specifically selected protein stabilizer/protectors which compositions are useful for aiding and protecting the skin, both generally and from specific ailments.

U.S. Pat. No. 6,551,361 to Cornwell et al. discloses methods of treating hair with a color protective composition. The patent is not directed to compounds which stabilize/protect skin protein. Similarly with EP 1,531,781, assigned to Cornwell et al. which is directed to hair treatment compositions comprising hydroxy compounds.

U.S. Publication No. 2004/0258717 to Sauermann et al. (assigned to Beiersdorf) discloses cosmetic and dermatological preparations containing creatine for treating and preventing dry skin and other negative alterations. The reference discloses a very wide range of molecules, including osmolytes (e.g., compounds which cells may accumulate when they are under dehydrating osmotic stress, e.g., high salinity, high evaporation, and which help to relieve such stress) which may be beneficial to skin. There is no teaching or suggestion of a means of selecting and no teaching or suggestion that compounds can be delivered as protein stabilizers/protectors.

Some products are sold which contain so-called “botanical osmolytes,” described as compounds which “reactivate natural water reserves of the skin, helping keep skin soft, radiant and healthy.” There is no disclosure of a test for selecting for protector compounds or of a method of treating skin ailments by stabilizing/protecting skin proteins.

### BRIEF DESCRIPTION OF THE INVENTION

In one embodiment, the invention relates to a method or test for selecting compounds which will serve as protein stabilizers/protectors for proteins found in skin, for example in the stratum corneum. Such proteins which may be stabilized may include structural proteins such as keratin, or enzymes involved in digesting protein linkages critical for the skin renewal process (e.g., serine proteases involved in desquamation process wherein desmosomal linkages between corneocytes are cleaved).

More specifically, the test involves utilizing enzymes which are part of the serine protease family (e.g., trypsin) in a model system to see what is the effect of certain tested compounds on the serine protease (e.g., the protective effect of compounds to prevent trypsin destabilization/denaturation). This protective effect can be measured as an increase in the half-life of the serine protease (e.g., of at least above 50%, preferably above 60%) when the tested compound(s) is/are used compared to if the serine protease is used without the protective compound(s).

Applicants have devised a test whereby they measure the rate of autolysis of the tested serine protease enzyme (e.g., trypsin). Compounds which enhance the rate of autolysis of the serine protease are more destabilizing (because autolysis occurs more quickly when enzyme is destabilized/denatured), and compounds which decrease the rate of autolysis of the tested protein (increase half-life) are more stabilizing (because autolysis occurs more slowly when enzyme is stabilized/not denatured).

In a second embodiment of the invention, the invention relates to a method of treating skin by delivering protein stabilizer to the skin. Because the linkage between improved skin and use of skin protein stabilizing compounds has not, to applicants knowledge, ever been made, this method of treatment is believed novel.

In a third embodiment, the invention relates to novel skin care compositions comprising protein stabilizers selected through the test described in the first embodiment.

These and other aspects, features and advantages will become apparent to those of ordinary skill in the art from a reading of the following detailed description and the appended claims. For the avoidance of doubt, any feature of one aspect of the present invention may be utilized in any other aspect of the invention. It is noted that the examples given in the description below are intended to clarify the invention and are not intended to limit the invention to those examples per se. Other than in the experimental examples, or where otherwise indicated, all numbers expressing quantities of ingredients or reaction conditions used herein are to be understood as modified in all instances by the term “about”. Similarly, all percentages are weight/weight percentages of the total composition unless otherwise indicated. Numerical ranges expressed in the format “from x to y” are understood to include x and y. When for a specific feature multiple preferred ranges are described in the format “from x to y”, it is understood that all ranges combining the different endpoints are also contemplated. Where the term “comprising” is used in the specification or claims, it is not intended to exclude any terms, steps or features not specifically recited. All temperatures are in degrees Celsius (° C.) unless specified otherwise. All measurements are in SI units unless specified otherwise. All documents cited are—in relevant part—incorporated herein by reference.

### BRIEF DESCRIPTION OF THE FIGURES

FIG. 1 is fluorescence spectra of freshly prepared solution of trypsin (1 mg/ml) at various time points. Iso-emissive points (fluorescence independent of time) are seen at wavelength values of 305 nm and 360 nm. Change in trypsin spectrum can be quantified by taking ratio, R, of the area between 320 nm and 330 nm; and the area between 380 nm and 400 nm.

FIG. 2 is graph of trypsin autolysis as followed by fluorescence spectroscopy in various different media (stabilizing compounds) with 0.1 M TRIS buffer (trishydroxymethyl aminoethane) at pH 7.5.

FIG. 3 is graph of trypsin autolysis as followed by fluorescence spectroscopy wherein media comprise stabilizing compounds as well as a destabilizing/denaturing force (e.g., sodium dodecyl sulphate).

### DETAILED DESCRIPTION OF THE INVENTION

In one embodiment, the invention relates to a method or test for selecting compounds, which will serve as protein stabilizers/protectors for proteins found in skin.

Specifically the test involves the use of serine protease (e.g., trypsin) in a model system for the testing of protein/enzyme stabilizing/protecting compounds. Serine proteases are used because desquamatory enzymes are part of a class of enzymes/proteins which, are serine proteases. Trypsin is also part of the serine protease family. Trypsin and desquamatory enzymes (although actual desquamatory enzymes present in the stratum corneum have been isolated and characterized, it is difficult to prepare in amounts sufficient for routine testing of protein/enzyme stabilizing compounds) are thought to exhibit a qualitatively identical dependence of their activity on water levels. Further testament to the relevance of trypsin in a model system is the fact that topical application of trypsin solution leads to a significant reduction of dry flakes at the skin surface of a person suffering from xerosis.

Serine proteases recognize a particular amino acid sequence in other enzymes/proteins and cleave the enzyme/protein at that particular site. Trypsin will also break itself down at biologically relevant concentrations.

The process of serine protease (e.g., trypsin) autolysis can be followed by using fluorescence spectroscopy. Trypsin, for example, contains three tryptophans in its amino acid sequence, and fluorescence properties of tryptophan change significantly when it is exposed to water relative to its non-polar environment in the folded and active state of trypsin. Consequently, the autolysis of trypsin and subsequent unfolding is accompanied by spectral changes that can be detected by obtaining fluorescence spectra at various time points after preparing a fresh solution of trypsin.

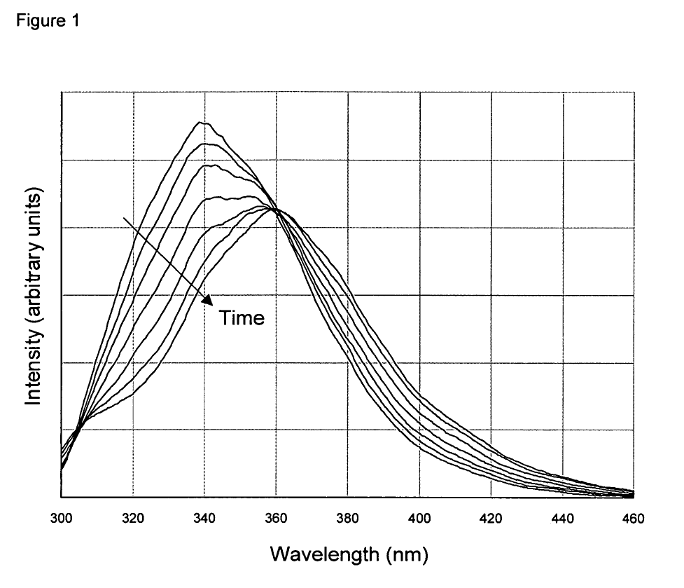

FIG. 1 displays fluorescence spectra of a freshly prepared solution of trypsin (1 mg/ml) at various time points. The iso-emissive points (fluorescence intensity independent of time) at 305 nm and 360 nm are typical of a situation where one fluorescent component (native protein) is the source of the other fluorescent component(s) (denatured protein and cleaved fragments). Applicants quantified the change in the trypsin spectrum by taking a ratio, R, of the area between 320 nm and 330 nm and the area between 380 nm and 400 nm:

$$R = \frac{{\int }_{320 nm}^{330 nm} ⅆ λ I ( λ )}{{\int }_{380 nm}^{400 nm} ⅆ λ I ( λ )} ;$$

wherein λ is wavelength; and

I is intensity of fluorescence.

The numerator represents the relative amount of native protein in the sample and the denominator represents relative amount of denatured protein and cleaved fragments. R therefore is a measure of how far the autolysis process has progressed. Although plotting either the numerator or the denominator would yield identical information, taking the ratio of the two is more accurate since the ratio does not depend on fluctuations in overall fluorescence intensity (e.g. those that may occur due to instrument fluctuations) that may occur during an experiment.

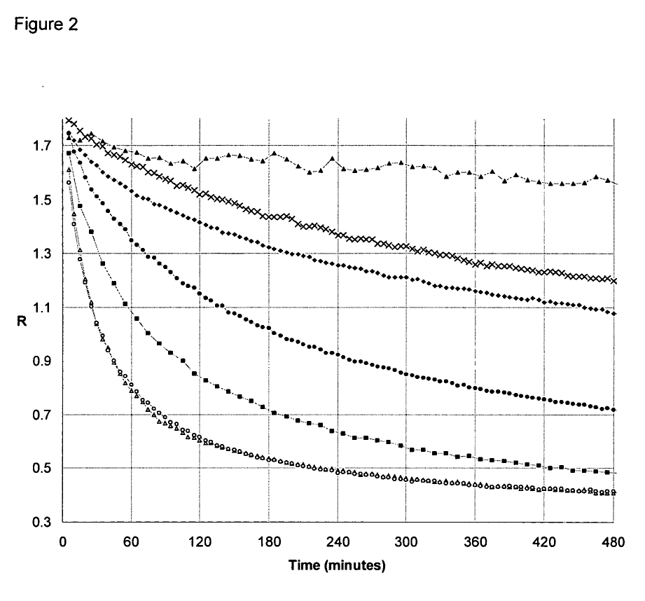

According to the first aspect of the invention, applicants have found that the ability of various compounds or combinations of compounds (e.g., the potential stabilizing compounds of the invention) to increase or depress the rate of trypsin autolysis can be compared by plotting R as a function of time as is shown, for example, in FIG. 2.

Thus, the invention specifically provides for a method of selecting a compound and/or compounds which can stabilize/protect enzymes/protein in the skin which method comprises:
(1) plotting the rate of hydrolysis of solution of a serine protease alone;
(2) plotting the rate of hydrolysis of the same solution when said serine protease is used in combination with a potential stabilizing compounds or compounds;
(3) comparing to see whether said potential stabilizing compound or compounds slow the rate of hydrolysis of the serine protease compared to the rate of hydrolysis when only a solution of serine protease is used (the change in rate is to some extent dependent on specific serine protease chosen as well as stabilizing compound(s) chosen; however, as noted below, there should be a decrease in rate which is measured or reflected by an increase in half-life of the serine protease); and
(4) selecting as a stabilizing compound or compounds such compound or compounds which provide a decrease in rate of hydrolysis of the serine protease when said compound is used with serine protease relative to the ratio of hydrolysis of serine protease alone, said decrease in rate of hydrolysis measured by an increase of at least 50% in the half-life of the serine protease.

While not wishing to be bound in any way, possible “stabilizing” compounds may include as follows:
(1) Amino acids and amino acid derivatives, for example: pyrollidone carboxylic acid (PCA), serine, glycine, arginine, ornithine, citrulline, alanine, histidine, urocanic acid;
(2) Alkylamines, for example methylamines, and more specifically trimethylamine N-oxide (TMAO) and 1-propanaminium, 2,3-dihydroxy-N,N,N-trimethyl-,chloride;
(3) Polyols, for example: glycerin;
(4) Sugars, for example: trehalose and sorbitol;

In a second embodiment of the invention, the invention comprises a method of treating skin wherein said method comprises applying about 0.1 to 25% by wt., preferably 0.5 to 20% by wt. of a skin protein stabilizing compound wherein said compound is defined by its ability to decrease rate of hydrolysis of the skin protein measured by an increase of at least 50%, preferably at least 60% in the half-life of serine protease when said stabilizing compound(s) is used with the serine protease compared to if the stabilizing compound is not used with the serine protease (i.e., serine protease used alone).

In one embodiment, the invention relates to a method of treating xerosis or a method of treating psoriasis comprising treating skin proteins with a stabilizing compound as defined above.

In a third embodiment of the invention, the invention relates to skin care compositions comprising skin proteins and skin protein stabilizers wherein said stabilizer is defined again by its ability to decrease rate of hydrolysis of skin protein (when measured with the stabilizing compound or compounds) relative to rate of hydrolysis of the skin protein alone, said decrease measured by an increase of at least 50% in the half-life of serine protease when said stabilizer is used with the serine protease compared to when the stabilizing compound is not used with the serine protease (i.e., serine protease used alone).

### EXAMPLES

Protocol—Fresh solutions of trypsin (2.5 mg/ml) were prepared by mixing the appropriate amount of solid trypsin from bovine pancreas (Sigma) with appropriate amounts of buffer solution (1M, pH 7.5; VWR), water, and protein stabilizing compound (pyrollidone carboxylic acid—Sigma Ultrapure; trimethylamine N-oxide—Fluka Purum; glycerin—J.T. Baker; 1-Propanaminium, 2,3-dihydroxy-N,N,N-trimethyl-, chloride—synthesized and purified in house), and/or protein destabilizing compound (urea—J.T. Baker Ultrapure Bioreagent); sodium dodecyl sulfate—ICN Ultrapure). All chemicals were used as supplied. Within minutes of dissolving the solid trypsin the solution was transferred into a quartz fluorescence cuvette and placed inside a Perkin Elmer LS 50 B fluorescence spectrometer. Fluorescence spectra were subsequently measured, either every 5 minutes or every 10 minutes. The excitation wavelength was 280 nm.

### Example 1

As noted in FIG. 1, and using the protocol set forth above, applicants plotted the fluorescence spectra of trypsin (1 mg/ml in 0.1 M TRIS buffer at pH 7.5) at various times after preparation of: 5 minutes, 30 minutes, 1 hour, 2 hours, 4 hours, 8 hours and 16 hours.

The iso-emissive point (fluorescence intensity independent of time) at 305 nm and 360 nm are typical of a situation where one fluorescent component (native protein) is the source of the other fluorescent component(s) (denatured protein and cleaved fragments). Application quantified the change in the trypsin spectrum by taking a ratio, R, of the area between 320 nm and 330 nm and the area between 380 m and 400 nm:

$$R = \frac{{\int }_{320 nm}^{330 nm} ⅆ λ I ( λ )}{{\int }_{380 nm}^{400 nm} ⅆ λ I ( λ )}$$

wherein λ is wavelength; and I is intensity of fluorescence.

### Example 2

A seen in FIG. 2, applicants plotted trypsin autolysis as followed by fluorescence spectroscopy in various media with 0.1 M TRIS buffer pH 7.5. These media include: 0.5 M PCA, i.e., pyrollidone carboxylic acid (closed triangles); 1 M TMAO (closed diamonds); 1 M glycerol (closed circles); 1M 1-propanaminium, 2,3-dihydroxy-N,N,N-trimethyl-,chloride (crosses); water (closed squares); 1 M urea (open triangles); and 0.5 mM SDS, i.e., sodium dodecyl sulphate (open circles).

The ability of various compounds or combinations of compounds to increase or depress the rate of trypsin autolysis can be compared by plotting R as a function of time as is shown, for example, in FIG. 2.

It can be observed that the addition of either 1 M urea or 0.5 mM of sodium dodecyl sulfate (SDS) causes a significant increase in the rate of trypsin hydrolysis. Both urea and SDS are well known for their ability to destabilize/unfold proteins and peptides. Since trypsin cleaves proteins/peptides (and itself) by the recognition of a particular amino acid sequence, the solvent exposure of that sequence will significantly contribute to the rate of cleavage. As the solvent exposure of proteins/peptides will increase dramatically upon (partial) unfolding, one can understand how the addition of a denaturant/destabilizer causes an increase in the rate of autolysis.

Since it has been shown that compounds known to destabilize protein/enzymes/peptides increase the rate of autolysis, it can be inferred that compounds that depress the rate of autolysis do so because they have a stabilizing/protecting effect on the protein. To test this hypothesis, and more specifically to predict the ability of specific compounds to stabilize/protect the skin enzymes responsible for desmosome digestion, applicants studied the effect of three different compounds—glycerol, triethylamine N-oxide (TMAO) and pyrollidone carboxylic acid (PCA) on the rate of trypsin autolysis.

Glycerol is well known for its ability to stabilize proteins/enzymes. For instance, it is common practice to store isolated enzymes in a concentrated glycerol solution to preserve activity. Glycerol is also a common ingredient in skin care products. Topical application of a glycerol/water mixture or a skin care formulation containing high levels of glycerol have been shown to alleviate xerosis, while it has been demonstrated that glycerol enhances corneocyte release in porcine skin ex vivo. Although the beneficial effects of glycerol have been explained in many different ways, none of the existing hypotheses take into account the ability of glycerol to stabilize/protect protein/enzymes other than through functioning as a skin humectant. However, FIG. 2 shows that glycerol significantly reduces the rate of trypsin autolysis, suggesting that it will have a stabilizing/protecting effect on desquamatory enzymes.

TMAO is a common and potent osmolyte. For instance, it is produced in large concentrations by deep sea animals to protect their proteins/enzymes from the denaturation as a consequence of the extreme pressure under which they exist. To applicants' knowledge, TMAO is not an ingredient in any commonly available skin care formulation. Yet, FIG. 2 shows that TMAO exceeds glycerol in its ability to stabilize trypsin against autolysis (i.e., line for closed diamonds is above line for closed circles).

1-Propanaminium, 2,3-dihydroxy-N,N,N-trimethyl-, chloride:

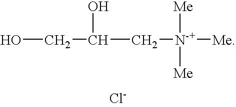

has a molecular structure similar to both glycerol and TMAO. Its ability to stabilize against trypsin autolysis was found to exceed that of both glycerol and TMAO (line with crosses above line with closed diamonds).

PCA is the major component of what is commonly referred to in the literature as the “natural moisturizing factor” (NMF). The NMF is a combination of amino acids and amino acid derivatives found in the upper layers of the stratum corneum. Up to 10% of the dry weight of corneocytes can be made up of NMF. The current view of the function of the NMF is that it is produced for its ability to draw water into the upper layers of the stratum corneum to ensure hydration, and consequently activity of the desquamatory enzymes. To applicants' knowledge, no reference has been made in the art to the ability of PCA or the NMF to stabilize/protect skin enzymes. Although, as an amino acid derivative, PCA is part of the wide class of compounds that potentially carry osmolytic properties, PCA is not specifically recognized as a potent osmolyte or protein stabilizer. PCA has been included in skin care formulations for its humectant properties. FIG. 2 shows that PCA is extraordinarily effective in stabilizing trypsin as can be inferred from the virtual absence of autolysis in the presence of trypsin (line with closed triangles above all others).

### Example 3

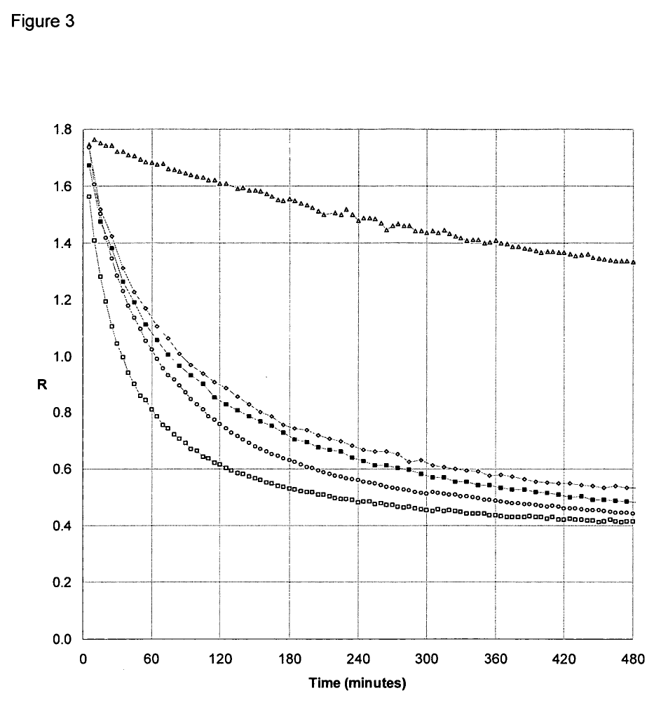

FIG. 3 shows that all of the compounds tested also are able to “protect” proteins against a denaturing force, in this case the presence of SDS at 0.5 mM concentration. Displayed are trypsin autolysis traces in the presence of 0.5 mM SDS and a protein stabilizer: 0.5 M PCA (open triangles); 1.0 M TMAO (open diamonds); 1.0 M glycerin (open circles). For reference purposes, also shown are autolysis traces in the presence of 0.5 mM SDS without any protein stabilizer (open squares) and without any SDS or protein stabilizer (closed squares).

All compounds cause a reduction in the rate of autolysis relative to a solution of SDS in buffer. For both TMAO and PCA the rate of autolysis in the presence of SDS is still slower than in the absence of both SDS and a protein stabilizer, i.e., SDS at a level of 0.5 mM is unable to reduce the efficacy of these compounds.

Finally, as the autolysis are carried out in aqueous solution, the humectant properties of the additives studied herein are not expected to play a role in their ability to reduce trypsin autolysis and, therefore, that effect can be exclusively attributed to the protein stabilizing/protecting abilities of the compounds tested. The fact that a compound, PCA, known to be produced in the stratum corneum itself to maintain regular desquamation, is found to have by far the strongest ability of any of the compounds tested to stabilize trypsin strongly supports the validity of trypsin as a model system for the desquamatory enzymes present in the stratum corneum. As such, the methodology presented herein appears to be an excellent method to assess the potential of any compound to stabilize skin enzymes.

1. Method of selecting or identifying a compound or compounds which can stabilize or protect protein/enzyme which method comprises:
(1) plotting the rate of hydrolysis of solution of a serine protease alone;
(2) plotting the rate of hydrolysis of he same solution when said serine protease is used in combination with a potential stabilizing compounds or compounds;
(3) comparing to see whether said potential stabilizing compound or compounds slows the rate of hydrolysis of the serine protease compared to the rate of hydrolysis when only a solution of serine protease is used;
(4) selecting as a stabilizing compound or compounds such compound or compounds which provide a decrease in rate of hydrolysis of the serine protease used with serine protease relative to the rate of hydrolysis of serine protease alone;
wherein said decrease in rate of hydrolysis is measured by an increase of at least about 50% in the half-life of the serine protease.

2. Method of treating skin comprising delivering to the skin a compound or compounds selected or identified by the method of claim 1.

3. A composition comprising compound or compounds selected or identified by the method of claim 1.

4. A method according to claim 1, wherein said decrease in rate of hydrolysis is measured by an increase of at least 60% in the half-life of the serine protease.
